(19) United States
(12) Patent Application Publication
(10) Pub. No.: US 2026/0181225 A1
(43) Pub. Date: Jun. 25, 2026

(54) METHOD FOR SETTING UP A VISUALIZATION SYSTEM AND MEDICAL VISUALIZATION SYSTEM

(71) Applicant: Schölly Fiberoptic GmbH, Denzlingen (DE)

(72) Inventors: Johannes BOURBON, Freiburg (DE); Christoph RAPP, Freiburg (DE); Pascal STRIEBEL, Denzlingen (DE); Michael SCHWÄRZLE, Denzlingen (DE); Markus KNOERZER, Freiburg (DE)

(73) Assignee: Schölly Fiberoptic GmbH, Denzlingen (DE)

(21) Appl. No.: 19/422,812

(22) Filed: Dec. 17, 2025

(30) Foreign Application Priority Data
Dec. 20, 2024 (DE) 102024139273.8

(51) Int. Cl. H04N 23/12; A61B 1/00; A61B 5/00; G06T 7/80; G06T 7/90; H04N 23/50; H04N 23/667; H04N 23/84

(52) U.S. Cl. CPC H04N 23/12; A61B 1/00009; A61B 1/00057; A61B 5/0077; G06T 7/80; G06T 7/90; H04N 23/667; H04N 23/84; G06T 2207/10016; G06T 2207/10024; G06T 2207/10056; G06T 2207/10068; G06T 2207/30004; H04N 23/555

(57) ABSTRACT

In order to provide a visualization system (1) that is tolerant of manufacturing-related fluctuations in sensor-detected signal intensities, a method for setting up this visualization system (1) is provided, which provides for an optical in-situ calibration measurement in which, with the aid of at least one image sensor (4) of the visualization system (1), calibration data (30) are sensed with the image sensor (4) as part of a respective optical calibration measurement. Based on this stored calibration data (30), the visualization system (1) can generate modified color channel signal values that are compensated in such a way that the manufacturing-related fluctuations no longer lead to a distortion of color ratios calculated from the modified color channel signal values. This approach thus enables a high degree of accuracy of the signal values and color ratios, in particular in multispectral imaging.

### CROSS-REFERENCE TO RELATED APPLICATIONS

This application claims priority from German Patent Application No. 10 2024 139 273.8, filed Dec. 20, 2024, which is incorporated herein by reference as if fully set forth.

### TECHNICAL FIELD

The invention relates to a method for setting up a visualization system, in particular an endoscopic visualization system, for advanced imaging, wherein the visualization system comprises at least one image sensor and several optical components and may also comprise an endoscope. The visualization system, more precisely its at least one image sensor, provides different color channels for multispectral imaging. The color channels are thus provided by an image sensor in each case. In other words, the at least one image sensor is set up for multispectral imaging, i.e., in particular, at least two image sensors of the visualization system (each) or at least one of two image sensors of the system can thus be set up for multispectral imaging. The optical components form at least one optical path that is used for multispectral imaging.

Furthermore, the invention relates to a visualization system as described above, which is designed for medical applications and can be configured, for example, as an endoscope, a microscope, or an exoscope. This system is designed in such a way that the aforementioned method can be implemented.

### BACKGROUND

In medical visualization systems such as endoscopes, illumination light generated by a light source is used to illuminate the surgical field. In this case, the illumination light from the light source first reaches the object to be observed, is reflected and/or scattered from there, and is then sensed by the visualization system, wherein the light passes through numerous optical components of the system until it finally hits an image sensor of the system in order to generate an image on a sensor surface of the image sensor. The optical components of the system typically comprise lens arrangements (which may also have certain spectral filter coatings) as well as beam splitters.

The manufacturing tolerances of such optical components are generally not negligible and can interact in such a way that there is considerable variation in the total transmission (through all optical components involved) of the optical path (which may be different in each case) used for the respective imaging, particularly in a specific sub-spectrum captured by the sensor. The transmission through the respective component depends on the wavelength of the light and can also vary across the captured image field. Relevant for the signals ultimately received at the image sensor is the respective (total) transmission through all components involved, which results in a respective spectral range and/or in a respective range of the sensor-captured image field.

Under certain circumstances, this variation can increase to such an extent that it exceeds the permissible tolerances of the visualization system and then prevents the system from achieving the required imaging accuracy in the specific application. This applies in particular to advanced visualization applications, in which the respective signal intensity in the respective imaging optical path must be detected by means of a sensor very precisely in a specific spectral range, wherein a variation in the total transmission of the optical path in the spectral range of interest that is not precisely known then acts as a disturbance variable.

In the prior art, for example, visualization systems are known that use a so-called white balance to improve color accuracy in image reproduction. For example, there are approaches that balance the respective signal strengths of red and blue color channels of an image sensor used for imaging in such a way that improved color reproduction is achieved. However, such white balance is only intended to ensure that the color white (and thus also shades of gray) is reproduced correctly. Such approaches typically only take into account the respective spectral range that is captured by the respective color channel of the image sensor (e.g., red/green/blue). Changes in intensity between the respective color channels can then be corrected by averaging during imaging. However, such white balance cannot compensate for variations that occur within a sub-spectrum captured by a color channel (e.g., within red wavelengths captured by a red color channel).

Especially when multimodal light sources or spectral filters or wavelength-selective beam splitters are used in imaging, each of which has a significant influence on the optical transmission of the respective optical path used for imaging and can thus significantly influence the intensity detected by the sensor, a simple white balance is often insufficient to achieve sufficient accuracy in the sensory detection of signal intensities in the respective spectral ranges. This applies in particular if the sub-spectrum to be detected extends over several color channels of the image sensor or forms only a sub-spectrum of a color spectrum that can be detected with one of the color channels. If a spectral shift occurs in the illumination light or a change in spectral transmission occurs in the optical components used due to manufacturing tolerances, these are not sufficiently taken into account during white balance, since only the white point is adjusted here, but the spectral ratios of individual sub-areas of the color spectra (sub-spectra) are not corrected correctly.

In the prior art, it is also known to determine respective color ratios from different signals of individual color channels of an image sensor (or, when using a beam splitter, also from color channels of several image sensors) of a visualization system, in particular for each individual pixel (or for corresponding pixels of several sensors). With the aid of an algorithm, conclusions can be drawn from such color ratios about the composition of the tissue or the concentration of a particular substance. This allows, for example, an index of a biomarker, in particular local oxygen saturation, to be determined with the aid of the algorithm.

For example, the algorithms used to calculate oxygen saturation are based on highly non-linear equations, wherein logarithmic values must be calculated. Therefore, depending on the algorithm used, the color ratios must be within a certain tolerance, which can sometimes be 2% or less, in order to ensure sufficiently high accuracy in determining the composition of the tissue, the concentration of a specific substance, or a calculated biomarker. However, as explained above, the light source used in imaging, including the light guide, the optical components such as lenses and beam splitters, and the image sensor used, may exhibit manufacturing-related variations that lead to fluctuations in the spectral distribution of the light used for imaging or to differences in spectral transmission. Such fluctuations can also cause the color channel signal values provided by each individual color channel of the image sensor to fluctuate significantly.

In the past, the approach was therefore often to define the specifications for the optical components with sufficient precision to achieve the required accuracy in determining the color channel signal values. However, this has the effect of making the production of the visualization system very expensive, for example because wavelength-selective thin-film filters can only be maintained within very narrow specifications with regard to spectral properties at great expense, or because only a correspondingly low yield can be achieved during production, or because components for generating the illumination light, e.g., LEDs or lasers, are subject to manufacturing-related fluctuations in their spectral properties, such as the dominant wavelength, and must therefore be selected at great expense in order to achieve the required tolerances.

### SUMMARY

Is therefore the object of the invention to provide a visualization system and an associated imaging method that is essentially immune to fluctuations in the sensor-detected signal intensities, whether these fluctuations are caused by manufacturing, temperature changes, or aging effects. The invention should also enable ad hoc compensation for such fluctuations. In particular, it should enable spectral resolution that allows individual sub-areas within the received/sensor-detected spectrum (sub-spectrum) to be distinguished, thereby enabling the determination of color relationships between individual spectral areas with the required accuracy. In general, signal intensities within individual sub-spectra should be detectable with high accuracy in order to reliably perform “advanced visualization” (e.g., determining the oxygen concentration in the observed tissue) or “advanced imaging” functions (e.g., high-precision fluorescence imaging) with the aid of (at least) one image sensor.

To solve this object, one or more of the features disclosed herein are provided according to the invention in a method of the type mentioned at the outset. In particular, the invention proposes, as a solution to the object, a method for setting up a visualization system in which a number N of optical calibration measurements are performed in a calibration mode with the visualization system, in each case using the at least one image sensor to capture calibration data. The number N may correspond to the number of different color channels of the visualization system or may be smaller than this number (so that a calibration measurement is not necessarily performed for each of the color channels).

In each of these N calibration measurements, a calibration light sub-spectrum is used which is designed for a respective spectral range to be corrected. The respective calibration light sub-spectrum is mainly captured by at least one associated color channel. This means that a calibration light sub-spectrum may also be captured by several color channels of the system, especially if it lies spectrally “between” two color channels. This is particularly useful for determining the respective signal amplitudes of the individual color channels and thus the “crosstalk” between these channels for the spectral range to be corrected, in order to ultimately determine suitable correction factors. The color channels involved in the invention can also be calculated (virtual) color channels, which are only obtained by offsetting signals from (physical/real) color channels of one or more image sensors of the system. For example, an RGB sensor physically only has the three color channels R, G, and B, but virtual color channels, for example for the intermediate color yellow, can also be calculated on the basis of signals from these three color channels. Such a virtual color channel then also covers only a sub-spectrum compared to the total spectrum that can be sensed by means of the color channels R/G/B.

The N calibration light sub-spectra differ from each other. Such a calibration light sub-spectrum can be generated, for example, by means of a multispectral light source, wherein individual color channels of the light source can then be activated to perform a calibration.

However, numerous other variants are also conceivable in which a broadband light source is used for calibration. In such cases, spectral separation can be performed in a beam splitter and/or by means of filter layers (for example, on the image sensor or in front of the image sensor). This method can also be used to correct signals from different color spectra. For example, a signal detected by an image sensor of the system attached to a beam splitter can be compared with a signal detected by another image sensor of the system attached to a second beam splitter (and thus receiving light from a different spectrum or in a different ratio). No narrow-band light source needs to be used for this purpose, because the spectral filtering or the generation of the respective calibration light spectrum is performed by spectrally selective optical components of the system (such as color filters or beam splitters) upstream of the respective image sensor. Furthermore, the signal ratios between two or more image sensors of the system can also be adjusted within the scope of the invention, wherein the image sensors can each detect overlapping or separate spectral ranges.

In calibration mode, at least one or more of the N different calibration light sub-spectra can be generated with at least one illumination light source of the visualization system, which is later used in the actual imaging; alternatively or in addition to this, the respective calibration light sub-spectrum can be generated by means of a separate calibration light arrangement, as will be described in more detail below.

The calibration data obtained in calibration mode (the calibration data can be determined from individual signals of the color channels by a computing unit, which may be part of the image signal processing unit) is stored in a memory of the visualization system.

It is also provided that, in a subsequent imaging mode (following the calibration mode), modified extended imaging will be performed using the at least one image sensor, in which signals from the at least one image sensor are processed in real time to produce live video image data. This may, for example, concern m<N different color channel signal values from a total of N color channels, for example when a VIS live image is generated from three of a total of five color channels R/G/B. “Modified” here means that the imaging deviates from a situation in which the calibration data is not available/not taken into account by the system.

At the same time, as part of a color channel signal correction based on the stored calibration data and on color channel signal values (in particular from n<N of N color channels of the system) output by the at least one image sensor, modified color channel signal values (within the scope of modified extended imaging) are calculated and, with the aid of these modified color channel signal values, extended image information is then determined (i.e., for example, based on two modified color channel signal values for a total of five different color channels).

This invention is advantageous for numerous applications, especially when signal intensities in a specific spectral range (and, consequently, the resulting signal ratios) must be recorded very accurately, which is often the case in advanced imaging applications.

Extended image information, such as the spatial distribution of a physiological parameter, can also be visualized by the system together with the live video image data. For example, at least one modified color ratio can be determined from the modified color channel signal values obtained in this way, which is further processed by the visualization system to display extended image information (such as local oxygen saturation). Here, too, modified means that the color ratio is different than before calibration. Thus, the modified color channel signal values do not necessarily have to be used for direct image generation (in the sense of directly modified image signals), but these values can also be processed in subsequent steps to form image information, which is then displayed as an image by the visualization system (e.g., in the form of a false-color image superimposed on a VIS image).

The entire spectrum of calibration light used in calibration mode is thus the sum of the individual calibration light sub-spectra; the calibration light sub-spectra may also overlap under certain circumstances. As mentioned above, the calibration light spectra used do not have to be spectrally separated when the light is generated (in the area of the light source); instead, spectral separation into the individual spectra can also take place in the optical path to the respective image sensor, for example by means of spectrally selective optical components such as filters, dichroic mirrors, beam splitters, and the like.

Based on the stored calibration data, an image signal used to display the extended image information can also be converted into a modified image signal by an image signal processing unit of the visualization system based on the modified color channel signal values, especially in real time.

In the approach according to the invention, several image sensors can also be used. In this case, a corresponding calibration measurement can be performed with each of these image sensors and/or with individual color channels of each of these image sensors. Such a procedure may be particularly useful if these image sensors/color channels are used in subsequent modified imaging and, optionally, temporarily changing lighting is also used.

Such temporarily changing illumination can be synchronized with a respective frame rate of the respective image sensor, wherein only a single image sensor can be used to alternately detect different spectral components. For different individual images (frames) captured by the visualization system at different recording times, spectrally different lighting can thus be used in each case. In this way, different spectral components can be captured at different times. For example, in imaging mode, broadband lighting can be temporarily superimposed on or replaced by spectrally narrow-band lighting.

A characteristic feature of the approach according to the invention may therefore be that, by designing the respective optical path (e.g., selection of optical filters, beam splitters, etc.) or by specifying the illumination used in the respective calibration measurement, a spectrum is captured with the respective image sensor, which is either only a partial spectrum of a spectrum that can in principle be captured with a color channel of the at least one image sensor, or a spectrum that can only be captured in total with at least two color channels of the image sensor. In contrast to a conventional white balance, the calibration data obtained with the respective calibration measurement can therefore refer to a sub-spectrum of a color spectrum of one of the color channels of the respective image sensor or, for example, to a color spectrum that extends over two spectrally adjacent color channels of the image sensor used in the (respective) calibration measurement. Often, the individual color channels of an image sensor are not sharply separated spectrally, but overlap. However, if spectrally narrow-band light falls on the image sensor, a spectral subdivision can be made by reading out the signal ratios between the individual color channels.

Furthermore, individual illumination light spectra can also be used sequentially in calibration mode to determine respective correction values, e.g., in the form of correction coefficients, as calibration data for the spectra of interest.

The respective calibration measurement, which can be performed during or after the manufacture of the visualization system (i.e., in particular shortly before each use of the imaging system), can thus, under certain circumstances, provide correction values as calibration data for each individual color channel of the visualization system. These correction values (as part of the calibration data) make it possible to correct for manufacturing-related variations in transmission along a respective optical path to the respective image sensor (more precisely, to the respective photosensitive element of the image sensor that senses the calibration light), but also for possible variations in the spectral characteristics of the illumination light source(s) used.

However, the correction is performed purely in terms of signal technology without any adjustment to the hardware, specifically solely by means of numerical compensation of the image signals, or more precisely, the color channel signal values that the respective image sensor delivers in the subsequent imaging mode. In this case, it is also possible that the color signal values are not supplied directly by a respective image sensor or by only one specific color channel of the image sensor, but that they are first calculated from signals from different color channels and/or from different image sensors. In any case, the result of this compensation is the aforementioned modified color channel signal values (which also include modified values previously calculated from color channel signal values), which allow for a significantly more accurate and/or stable determination of color ratios in particular. The calibration mode can also be divided into several individual measurements, in which case individual optical components of the system are measured. But even then, the result is calibration data that is sensed by at least one image sensor, which (optionally together with further calibration data determined previously in individual measurements) allows conclusions to be drawn about the actual spectral properties of the fully assembled visualization system.

In medical applications, it is often the case that the finished visualization system, which may comprise, for example, an endoscope and an associated camera head, is only fully assembled shortly before a surgical operation. This is because the individual components are typically stored in sterile plastic packaging and only assembled into the complete system shortly before the actual operation, wherein different endoscopes with different camera heads may be used depending on the operation. In such a situation, the user can perform the calibration mode and capture the calibration data themselves with the fully assembled visualization system. For example, the calibration mode can be performed with a color image sensor of a camera head in order to capture calibration data sensorically and store it in the aforementioned memory. This calibration data can then be taken into account in subsequent advanced video imaging performed with the visualization system. Such calibration can therefore be performed quickly and reliably, even by untrained personnel.

The calculation of the modified color channel signal values can be performed by an image signal processing unit of the visualization system, in particular as part of a camera control unit of the visualization system, preferably in real time. This image signal processing unit can also be set up to determine and store (in an internal or external memory) the calibration data from image signals of the image sensor in calibration mode. The calibration mode can also be performed only once during the manufacture and assembly of the visualization system, or several times, even after the assembly is complete.

The aforementioned memory is preferably an internal memory of the visualization system. However, it is also possible for the calibration data to be stored in an external memory, for example in a cloud, in which case the visualization system accesses this external memory in imaging mode or before imaging mode in order to transfer the calibration data to an internal memory (this can also be a volatile memory). Depending on the design of the system, the entire processing can even be carried out in a cloud, so that internal memory for the calibration data is completely unnecessary.

Performing such in-situ calibrations at the system level according to the invention also has the advantage that, for example, a service technician or a surgeon using the visualization system can easily perform such calibration measurements, especially after replacing a light source or a camera head of the system and/or at regular intervals of a few months or before each use, in order to always be able to store updated calibration data in the aforementioned (internal or external) memory of the system. In this way, aging effects of the light sources, but also temperature effects or effects due to the storage of system components, which each have an impact on the spectral properties of the respective component, can be compensated for. It should be noted that even minor deviations in the course of the optical path, such as optical components that shift slightly in space, can have a significant effect on the spectral transmission of the respective optical path, as this often depends on the angle of incidence of the respective light beam. Such effects can also be compensated for by the approach according to the invention.

Unlike white balance, the approach according to the invention may in particular provide for no broadband light spectrum being detected during the calibration mode, but instead a (respective) calibration arrangement is used which provides spectrally narrow-band calibration light with the aid of a suitable target (which only reflects narrow-band light), as will be explained in more detail below. However, spectral selectivity in the calibration measurement can also be achieved with broadband illumination by using wavelength-selective optical elements such as spectral filters or beam splitters in the calibration arrangement (these elements can be part of the visualization system itself or part of a calibration light arrangement used in calibration mode).

The invention thus proposes a sophisticated calibration mechanism with which spectral disturbance variables can be numerically compensated so that the sensory detection of signal intensities performed with the visualization system remains within the accuracy required for advanced visualization applications. The invention does not address the actual algorithm on which the respective advanced visualization application is based, but aims to provide the input data processed by an algorithm such as described above with higher accuracy; even if the visualization system used exhibits typical manufacturing-related fluctuations in transmission that are not negligible, or if individual emitters exhibit such fluctuations in their spectral distribution.

An advantage here is that certain variations in the manufacture of individual components of the visualization system can be tolerated (i.e., the corresponding tolerances can be interpreted more broadly), so that less restrictive/strict requirements need to be specified because these variations can be compensated for with the approach according to the invention. The solution according to the invention thus enables effective correction of manufacturing-related variations in the optical path used for imaging. Overall, this greatly reduces effects that have a negative impact on the accuracy of determining color ratios.

As explained, the approach according to the invention consists in achieving the necessary spectral compensation of manufacturing-related fluctuations in the total transmission in the respective optical path (used for imaging) and/or comparable fluctuations in illumination, preferably exclusively by means of signal processing. This means that the fluctuation in transmission is not eliminated by adjusting the hardware, but is taken into account by intelligent signal processing in such a way that it no longer (or at least less) interferes with the acquisition of the image signals or signal intensities. For this purpose, it may be provided in particular (especially within an image processing chain of the visualization system, which is designed and set up to generate a continuous video image data stream, known as a “video chain”) that a signal processing step is included that takes into account a manufacturing-related spectral variation (as described above) within the optical path and compensates for it accordingly by modifying the signals of the image sensor used for imaging (in particular by respective signal amplification or signal attenuation). Such an (additional) signal processing step can be implemented in software, for example, by means of a “calibration block.”

The solution according to the invention can be implemented in particular in a multi-step process as follows:

1) Spectral transmission data is measured as spectral calibration data for the respective optical path to be compensated. (For such a calibration measurement, a wavelength-selective element, e.g., a diffraction grating, an optical filter, or a reflective target, can be used, which selects a sub-spectrum from the spectrum emitted by the light source (in transmission or, for example, in reflection) in order to provide the respective calibration light sub-spectrum); and/or

2) a spectral shift of a light source used in subsequent imaging is measured as spectral calibration data; such a shift may be due, for example, to aging effects of the light source.

3) Building on steps 1) and/or 2) and based on the spectral calibration data obtained from measurement data obtained from this, a calculation is used to calculate the respective signal attenuation or signal amplification of the respective color channel of the respective image sensor used for imaging (several image sensors may also be used) and, preferably, also a respective crosstalk between the individual different color channels of the (respective) image sensor. The signal modifications to be implemented can be stored, for example, in the form of compensation data. In particular, any spectral shift in the emission characteristics of the light source used/to be used in subsequent imaging can be taken into account. Such changes can be detected by step 2), in particular by comparing signal values that have been recorded for several spectrally limited bands. Such a spectral shift of the light source may be due to drifts or, for example, manufacturing tolerances.

Step 3) can thus be used to determine numerical compensation values/data for each color channel of the color image sensor used. The color image sensor can be, for example, a classic RGB sensor or an image sensor equipped with a combination of color filters (e.g., CYGM or RGBW), or a multispectral image sensor with a higher number of color filters, e.g., with 16 different color filters. Advantageously, such data can be captured using suitable measurement technology at a resolution in the sub-RGB range (i.e., with higher spectral resolution compared to that offered by a typical RGB sensor). The compensation values can be calculated on a pixel-by-pixel basis or for individual areas of pixels of the image sensor (so-called “pixel binning”).

Calculated signal levels, which are only obtained by processing signal values from multiple color channels, can also be corrected using such compensation data. This approach is also described here using the term “sub-spectrum” because the actual color channel has a higher spectral width. Thus, the proposed method can also be used to correct values calculated from color channel signal values (after they have been calculated) in order to obtain “modified color channel signal values” within the meaning of the invention.

4) The image signals of the image sensor can be modified or corrected (especially in the form of raw signals), preferably before the actual signal processing into video image data, specifically with the help of the numerical compensation values/compensation data measured in steps 1) and/or 2) and/or calculated in step 3). This signal modification can be performed, for example, using the described correction module (“calibration block”). The correction module can be implemented in separate hardware or by means of software.

The numerical compensation values can be recorded as calibration data, for example in the form of amplification or attenuation factors. Such factors can then be applied to the signal of the respective color channel (or to quantities calculated from it as mentioned above) in order to obtain/calculate a compensated signal/compensated calculated quantity (both collectively referred to as “modified color channel signal values”). In such a compensated signal/calculated value, manufacturing-related fluctuations are thus already taken into account or compensated for.

The innovative approach of the invention thus lies in detecting a spectral shift/drift of a) a light source used for imaging and/or b) an optical transmission along an optical path to the respective image sensor (more precisely, to the respective photosensitive element, which may include, for example, color filters), each as a function of wavelength, within the scope of a respective calibration measurement using the image sensor. This means that the optical components of the visualization system do not necessarily have to be measured/characterized before assembly, and the respective calibration measurement is performed with the fully assembled visualization system. Since such shifts can occur over time, it is advisable to perform the method according to the invention on an ad hoc basis in order to always have up-to-date calibration data available.

In this way, as explained above, corrected and thus more accurate image data, in particular more accurate (spectral) intensity values, can be sensed with the respective image sensor of the visualization system during subsequent imaging (which is then based on the calibration performed previously). For this purpose, the image signals of the image sensor are modified on the basis of the calibration data previously measured using the visualization system. This modification can be carried out in real time, in particular by means of signal processing. Particularly when using optical components with very high wavelength selectivity, such as beam splitters, this approach according to the invention, in particular the consideration of spectral shifts in the respective transmission properties of the respective optical component of the visualization system, can significantly increase the efficiency and accuracy of the proposed imaging.

The object mentioned at the beginning can also be solved with further features according to the description and claims that follow, which are explained in more detail below:

The calibration data may in particular comprise (under certain circumstances respective) color channel correction values which describe which target deviation the respective calibration light sub-spectrum generates in the assigned color channel and/or crosstalk correction values that describe spectral crosstalk of the respective calibration light sub-spectrum to at least one adjacent color channel.

In contrast to conventional white balance, the approach according to the invention aims to specifically compensate for individual spectral ranges that are subject to manufacturing-related fluctuations using signal technology in order to be able to capture modified and thus more accurate spectral measurement data with the respective image sensor (in the case of combined signals, possibly also with several image sensors). Accordingly, the calibration data for different optical paths (which are used in the visualization system for the respective imaging) and/or for different color channels (these can be formed, for example, in the case of spatial separation of spectral components by beam splitters or at the pixel level, or also temporally, for example in the case of changing spectral illumination) can be recorded separately in each case. This allows path-dependent and/or color channel-dependent compensation parameters to be calculated. Such compensation parameters, which may be available as color compensation values, for example, can then be used to generate the modified color channel signal values (as explained, these may also be quantities calculated from color channel signals) in order to enable highly accurate, in particular multispectral, imaging. Accordingly, a visualization system according to the invention, more precisely its image signal processing unit, can also be set up to capture the aforementioned calibration data for different optical paths and/or for different color channels separately (sensorily with the respective image sensor).

For example, the imaging mode may comprise sub-spectral signal processing of the modified color channel signal values. Such sub-spectral signal processing can be used to obtain sub-spectral image information that represents only a portion of the overall spectrum used for imaging, i.e., in particular, a portion of the illumination light spectrum of the illumination light source. In principle, the correction according to the invention can also be applied to color channels through which no illumination light from a light source is detected, but only, for example, fluorescence light.

In the context of such sub-spectral signal processing, in addition to initial information directly detected by the sensor (for example, in the form of a white light image and/or a spectral image, in particular a fluorescence image), additional information, in particular a spatial distribution StO₂ (x,y) of an oxygen saturation StO₂ can also be calculated from local color conditions based on the modified color channel signal values.

Such local color ratios CR(x,y) can be calculated from respective modified color channel signal values (e.g., R_{mod}(x,y), G_{mod}(x,y), B_{mod}(x,y)) of respective different color channels (R/G/B) of the at least one image sensor of the visualization system. A color signal value can also be obtained from the calculation of color signal values of one or more image sensors, and the correction values can be applied to such calculated values.

In sub-spectral imaging, a monochrome image sensor can also be used, which only senses a specific sub-spectrum (e.g., in the NIR) that is of interest, but not the entire wavelength spectrum used for imaging (e.g., VIS+NIR). In addition, as mentioned above, the approach according to the invention can also be used in the case of temporarily changing spectral illumination.

In the context of sub-spectral signal processing, a sub-spectrum of a total spectrum sensed by the visualization system for imaging can also be sensed separately, i.e., in particular spatially or temporally separated from the total spectrum, in the form of sub-spectral image signals using at least one image sensor and processed into modified sub-spectral image signals by an image signal processing unit, in particular the one mentioned above, based on the calibration data. This approach is suitable, for example, for fluorescence imaging, the accuracy of which can be improved with the approach according to the invention.

The calibration data mentioned can, for example, describe wavelength-dependent transmission properties of at least one optical path used for imaging within the visualization system and/or a spectral emission characteristic of an illumination light source used in the subsequent imaging mode.

In calibration mode, at least one calibration light arrangement can be used to perform the respective optical calibration measurement. Such a calibration arrangement provides a calibration light sub-spectrum whose half-width can be limited to less than 50 nm, preferably less than 30 nm, or even <10 nm, or can be actively limited (using appropriate optical components). In this way, it is possible (by adjusting the respective calibration arrangement or by using several such calibration arrangements) to capture respective spectral calibration data (i.e., in particular, respective correction values for several color channels of the visualization system used in imaging mode) spectrally selectively for several different spectral ranges (=respective calibration light spectrum). A half-width here can be understood as a “full-width-half-maximum” (FWHM) value of the respective spectral curve of the emission light.

The light spectrum used for calibration can either be provided by a light source itself, or a target can be used which is illuminated by the light source with a more or less broadband spectrum and then reflects/transmits/emits a correspondingly limited spectrum. These two basic options are described in more detail below:

At a minimum, a calibration light arrangement can be implemented using a passive optical target and also include a broadband light source (this can be, in particular, an illumination light source of the system). Such a target (in particular, the respective target) can re-emit or transmit a comparatively narrow spectrum with a half-value width of less than 50 nm when irradiated with suitable broadband light. For this purpose, the passive optical target may have at least one wavelength-selective optical element, for example an optical grating, a thin-film filter, or several different types of quantum dots (each of which may then have its own narrow-band emission characteristic), to name just a few examples. “Narrow-band” can generally be understood here to mean that the corresponding full width at half maximum (FWHM) of the characteristic is less than 100 nm, less than 50 nm, or even less than 30 nm. When diffraction gratings are used, the FWHM can also be less than 15 nm, or even <10 nm.

Such a target is therefore not a light source in the true sense of the word, but re-emits narrow-band calibration light only in response to irradiation with (typically broadband) illumination light from a conventional illumination light source. In contrast to conventional targets such as those used for white balance, the spectral width of the wavelengths emitted by the passive optical target and ultimately detected by the visualization system in calibration mode is narrowly limited to a few 10 nm. This makes it possible to record the respective spectral correction values for individual spectral ranges with high precision.

A passive optical target within the meaning of the invention can also be obtained if the target has a (highly) wave-selective element. Such a wave-selective element transmits (e.g., in the case of a thin-film filter), reflects, scatters, or re-emits illumination light falling on the target only within a very narrow spectral bandwidth of less than 50 nm, preferably less than 30 nm, particularly preferably less than 10 nm, or even less than 1 nm. Such a wavelength-selective element can be realized, for example, by an optical grating (e.g., realized by means of a chrome mask on a glass substrate) or with quantum dots.

The respective wavelength-selective element can also be based on a plasmonic effect. One or more light sources of the visualization system can be used as the illumination light source that illuminates the target and thus generates the calibration light (in-situ approach), or at least one separate light source can be used for this purpose (still in-situ, since the entire imaging optics of the visualization system continues to be used for the calibration measurement).

Another possible design provides for one type of the aforementioned different types of quantum dots to be excited to emission by alternately switching on and off several different illumination light sources of the visualization system at different times. Accordingly, the target then emits a precisely defined bandwidth of wavelengths very specifically at the respective switch-on time. This makes it possible to sequentially capture the desired spectral correction data as part of the calibration data using the visualization system. In such a calibration, the visualization system “looks” at the passive target and captures the calibration light wavelengths emitted by it.

As an alternative to the passive target mentioned above, a calibration light source (which may be spectrally tunable) can also be used in calibration mode, e.g., a light source that is already calibrated and can therefore emit a precisely defined intensity within a precisely defined wavelength range of preferably less than 50 nm (FWHM), particularly preferably of <30 nm or even <10 nm. Such a calibration light source can be implemented in particular with an ECDL (“external cavity diode laser”) or by means of several separate lasers.

As explained above, the approach according to the invention allows the respective light source of the system that is also used in the subsequent imaging mode to be used in calibration mode; however, this is not mandatory, and separate calibration light sources may also be used.

In addition, in calibration mode, measured values can be recorded using at least one image sensor, which later generates the image signals, in particular video image data, in imaging mode. The calibration performed with this image sensor can thus provide signals for advanced visualization, i.e., in particular for a subsequent calculation step. The decisive factor here is to use the visualization system in calibration mode to accurately detect the respective spectral transmission for the individual wavelength ranges that are to be detected by each of the system's different color channels. This enables the precise calculation of the modified color channel signal values/modified image signals that form the basis for advanced visualization.

With a total of five LED light sources, for example, one calibration measurement can be performed per color channel, with only one of these five LEDs illuminating the target. Accordingly, at least one wavelength-selective element can then be used on the target per color channel/per narrow-band illumination light source (e.g., five different types of quantum dots). It is understood that five different targets could also be used. Depending on the bandwidth of the LED, it is not necessary to use a wavelength-selective target. If the LEDs are narrow-band enough for the signals to be identified, a simple reflective target can also be used. In particular, when using laser light sources, a wavelength-selective target is usually not necessary.

The use of color charts, which typically have an emission bandwidth of several 100 nm (i.e., with reference to the light emitted by the color chart), would not be suitable for the calibration method according to the invention because it would not allow the respective transmission along the optical path to be detected with sufficiently high selectivity within a respective very narrow spectral range using the at least one image sensor of the system.

One of the advantages of the invention is therefore that, even with a sensor element that is not particularly spectrally selective, namely the at least one image sensor, each of the color channels can nevertheless be measured very accurately in terms of intensity values/transmission along the optical path in calibration mode, because the calibration light sub-spectrum is designed accordingly for the spectral range to be detected/corrected.

In imaging mode, numerical color channel compensation values can also be determined from the calibration data. With such values, different color channel signal values output by the at least one image sensor, or quantities calculated from such color channel signal values, can be converted into “modified color channel signal values.” In this way, it is possible in particular to determine at least one modified color ratio.

In calibration mode, it is possible in particular to capture spectral transmission data from a (respective) optical path used for imaging in imaging mode using the calibration data. Depending on the design of the visualization system, it may be advantageous if, in calibration mode, spectral transmission data from at least two separate optical paths within the system are acquired by the calibration data (in particular within the scope of a calibration measurement and using one of the calibration light sub-spectra), for example when two image sensors are used. The at least two different/separate optical paths may refer to sub-paths, such as those created when using beam splitters. This means that the respective calibration measurement can be performed for different image sensors that receive light from a beam splitter, each with the same calibration light sub-spectrum, in order to capture the respective calibration data for this calibration light sub-spectrum for each image sensor and thus optical sub-path.

However, the calibration data may also differ in terms of wavelength and/or signal processing. For example, if a blue spectral range is sensed by a first image sensor whose color channel signal values are used to calculate extended image information (using a corresponding imaging algorithm), a signal from a second image sensor that also receives light from the beam splitter may be a mere artifact. However, this parasitic signal can also be included in the calculation of the extended image information, for example, if the calibration measurement shows that there is significant optical crosstalk in the relevant wavelength range to the second image sensor.

In particular, the visualization system may be designed with a first optical path for white light imaging (WLI) and a second optical path for sub-spectral imaging, for example in the NIR wavelength range. The first optical path may end at a color image sensor and/or the second path may end at a monochrome image sensor or at a color image sensor. The first and second paths may also overlap at least partially (e.g., if both pass through a shared lens) and may be spatially separated from each other, for example, by means of a wavelength-selective beam splitter. However, it is also possible to separate the two paths at the pixel level (for example, when using an RGBX sensor) or by means of optical filters attached in front of the respective sensor. Furthermore, the separation can be performed temporally, in which case both paths can be identical but are read out at different times. In such cases, the calibration measurements can provide calibration data for both optical paths, and calibration data can be recorded and stored for each of the two optical paths, in particular for each calibration light sub-spectrum used.

In calibration mode, the spectral emission characteristics of an illumination light source used in the subsequent imaging mode can also be recorded by the calibration data. This can preferably be done by using narrow-band calibration light emitted (e.g., re-emitted or transmitted or scattered) by at least one wavelength-selective optical element, in particular a passive optical target as described above, to determine the calibration data.

The visualization system may also comprise an endoscope/exoscope. In such a case, it is advantageous if an optical calibration device specially designed for the endoscope/exoscope is used in the calibration mode. Here, the calibration device can be pushed onto a shaft of the endoscope/an optic of the exoscope while the optical calibration measurement is performed with the visualization system.

The calibration device can, for example, be designed in the form of a sleeve that can be pushed onto the endoscope shaft; it can also comprise a passive optical target as described above. This target can emit a respective calibration wavelength to be measured as soon as it is illuminated with light, in particular from one of the light sources of the system. The main advantage of this approach is that, after the calibration device has been placed on the endoscope shaft, the target can be reproducibly positioned at a precisely defined distance from an imaging optic of the visualization system without the need for time-consuming manual alignment.

Preferably, during the optical calibration measurement, a respective light source of the visualization system (e.g., the respective LED mentioned above) can illuminate the target/calibration device so that it can emit a calibration light in a precisely defined wavelength range (=calibration light sub-spectrum) toward the imaging optics.

The calibration device described can be designed in particular in the form of a miniaturized integrating sphere into which the illumination light is introduced.

In the methods described above according to the invention, previously known approaches of “debayering”/“demosaicing” can also be used to generate the color signal values. With such approaches, the spatial resolution per color channel can be artificially increased, wherein identical color filters at the pixel level form a common color channel. For example, a typical 2×2 Bayer pattern has two green pixels, one red pixel, and one blue pixel, which can be assigned to a green, red, and blue color channel, respectively. According to the invention, a narrow-band calibration light used in calibration mode can be detected by several such color filters at the pixel level, which improves the accuracy of the desired calibration. For example, in the calibration measurement, more precisely in the calibration data, it is possible to detect the signal level that a calibration light in the red wavelength range generates on the green and blue pixels (=crosstalk) in order to take these effects into account. This makes it possible to filter sub-spectra that only cover a sub-range of a color channel's spectrum from the spectral curve by calculating the different color channels and to identify their signal amplitude in order to use them for “advanced visualization.”

According to the invention, the respective color channel of the visualization system can be implemented in numerous ways: for example, an RGBX sensor or a hyperspectral sensor can be used. It is also conceivable to use a color image sensor to detect an IR wavelength, i.e., to capture monochromatic images at this IR wavelength with the three color channels of the color image sensor. This is because, with the help of the calibration method according to the invention, the variations in sensitivity of the IR wavelength to be captured between the individual color channels/color filters (at pixel level) of the color sensor can be corrected quickly and efficiently using optical calibration measurement by capturing corresponding correction values as part of the calibration data. As a result, a respective IR intensity can be recorded with e.g. high precision in all pixels, even though red, blue, and green pixels detect the IR wavelength.

The invention is generally advantageous not only in applications such as determining local oxygen saturation as an example of a biomarker, but also in all imaging procedures in which, in the sense of “advanced visualization,” additional information is calculated from color ratios detected by sensors, which can be visualized to the user, ideally in addition to normal white light imaging. This can include, for example: fluorescence imaging; visualization of nerve tissue; visualization of certain tissue types using biomarkers. The approach according to the invention offers decisive advantages in all these areas of application.

In a specific application of the invention, it may be the case that the actual correction/modification of the color channel signal values or image signal data using the calibration data/correction values determined in the calibration measurement takes place in real time, but not necessarily within the visualization system. For example, the visualization system could also supply raw data that is transferred in real time to a cloud, where the image signals are then modified in accordance with the invention using the calibration data/correction values determined beforehand and, if necessary, also stored in the cloud. The cloud can then return signal-corrected measurement/image data, which is then processed by the system, for example to determine blood oxygen saturation. The calculation of such extended image information can also be performed directly in the cloud, and the data/images calculated in this way can be sent back to the visualization system for display/visualization.

The modification/correction of the raw signals supplied by the respective color channel of the sensor according to the invention can take place before or after “debayering”. Depending on the size of the correction, image signals may be clipped. Since this would distort the signals, it can be compensated for by appropriate scaling.

Furthermore, according to the invention, it is also possible to use the calibration data to capture certain correction values for a specific area or for individual pixels and to simply calculate the remaining correction values; in this way, further correction values can be calculated for an overall image, for example on the basis of a specific assumption, by means of extrapolation. In this way, a spatially resolved correction matrix could also be obtained, in which each individual image pixel is assigned a corresponding correction value based on the calibration data in accordance with the invention. In this way, modified color channel signal values or image signals can also be calculated in accordance with the invention.

Since individual transmission values along the respective optical path within the system are not added together but rather multiplied to give the total transmission, correction values according to the invention (which were determined on the basis of the optical calibration measurement) can be understood in particular as a relative amplification factor that corrects a respective (production-related) relative offset (preferably spatially resolved) of the respective color channel. The correction values can therefore be designed as pre-factors, but in the form of thresholds that are subtracted from the respective signals. In both cases, the correction values do not have to be applied directly to the individual color signals, but can also be applied to signals that have already been calculated with each other, for example, as a prefactor of ratios between color channels or as a coefficient in polynomial equations or as an exponent. However, even in such cases, modified color channel signal values can be obtained within the meaning of the invention.

In addition, an exemplary design of a visualization system according to the invention with four color channels (R, G, B1, B2) and a total of four different narrow-band light sources is described here: In such a situation, four calibration measurements must be performed, wherein in each calibration measurement only one of the light sources is used to illuminate the described passive optical target. When using a red light source, a correction value can be sensed for a red color channel. This makes it possible to correct the deviation of the optical transmission along the image path in the red wavelength range (in relation to the ideal design data of the overall system) in terms of signal technology during subsequent image signal processing (=red color channel compensation or color channel correction value).

In addition, when using the red calibration wavelength, a respective signal can also be obtained with the three other color channels as part of the optical calibration measurement. Such measurement signals can be used in calibration mode to calculate so-called crosstalk correction values. These indicate the proportion of the signal that is incorrectly received in the other color channel instead of the red color channel.

Thus, with an optical calibration measurement according to the invention, not only color channel-specific color compensation values can be determined, but also color channel-specific crosstalk correction values, which can be taken into account in the calculation of modified image signals during the imaging mode. This further increases the accuracy of the imaging.

If, for example, a spectral shift occurs in the emission characteristics of the illumination light source used during one of the calibration measurements, causing its emission wavelength to deviate slightly from the specification, this can result in an increase or decrease in the corresponding color channel-specific correction value and/or in a certain amount of crosstalk (optical interference) in a neighboring color channel. In any case, however, the crosstalk will change. It is therefore preferable if, in calibration mode, not only the respective correction values for each individual color channel (color compensation values) are recorded by the sensor, but also the respective crosstalk correction values of the remaining color channels. Depending on the application, however, it may be sufficient, for example, to ignore all of these crosstalk correction values or to consider only a single one (e.g., that of a spectrally directly adjacent color channel). Accordingly, it may be provided that, in imaging mode, at least one color channel-specific color compensation value, which is based on the calibration data, and/or at least one color channel-specific crosstalk correction value, which is based on the calibration data, is/are taken into account when determining the modified image signals.

In contrast to a conventional white balance, the color channel-specific color compensation or correction values according to the invention, which can be applied to individual color channels, are recorded separately from the corresponding color compensation or correction values of the other color channels in each case using a respective optical calibration measurement with the visualization system. With this approach, relative color ratios in particular can be recorded with high precision in the subsequent application with the at least one image sensor, because the manufacturing-related fluctuations along the optical path are compensated for in each case with the help of the correction values.

In addition to the singular detection and compensation of correction values of individual color channels, a further difference may be that the correction values are detected in a narrow band. This means that correction values are only determined for a sub-spectrum of a spectrum that can be detected with a color channel or, for example, for individual sensors that only detect a specific spectral section via beam splitters and/or filters. The calibration light sub-spectrum used to determine such a correction value may also lie in the non-visible wavelength range. Furthermore, the recorded correction values do not necessarily have to be used to correct the display of images, but rather for the modified calculation of signal levels or measured values that are relevant for “advanced visualization.”

Taking the crosstalk correction values into account further increases accuracy, as it also makes it possible to correct effects that are attributable to spectral crosstalk on the other color channels.

A typical image signal processing chain used in the visualization system might look like this: First, analog signals (for each individual pixel) are generated on the (respective) image sensor during imaging and converted into digital signals by an ADC on the image sensor chip. The correction values can only be applied before the processing steps/blocks of the advanced visualization solution that are relevant for the calculation of signals or measured values, while the actual WLI image chain can remain unaffected. The signal correction sought by the invention can thus preferably only take place in the imaging path in which the calculations for advanced visualization take place. Since RAW data is preferably used here, it can be derived separately before further processing in the WLI path. The color channel signal correction according to the invention can thus be applied to the described digital raw signals of the image sensor.

In calibration mode, however, it may be provided that de-bayering is first performed before the respective correction values (e.g., as respective relative amplification factors) are determined as part of the calibration data. During the subsequent generation of a continuous video image data stream in imaging mode, the raw digital signals from the image sensor can then be amplified or attenuated accordingly using the correction values, either before or after DE-BAYERING, and thus numerically corrected in order to obtain the modified color channel signal values or modified image signals according to the invention. In the simplest case, it is conceivable that only a single color channel, which is used for “advanced visualization,” for example, is corrected using the method according to the invention. In this case, there is only a single calibration measurement and only correction values for a single color channel, even if the image sensor still comprises several color channels. The correction may also relate, for example, to a color channel of an additional sensor that is connected via a beam splitter and is set up to capture a specific sub-spectrum, particularly in the non-visible wavelength range.

According to a further design, it may be provided that in calibration mode, the calibration data is captured with spatial resolution for different image areas and/or for different pixels and stored in the form of location-dependent correction data. In this way, for example, meaningful compensations can be made if, due to a defective light guide, image field-dependent variations in illumination occur in the field of view of the visualization system. In order to compensate for such effects, the color channel signal values for different image pixels can be modified differently in imaging mode as part of color channel signal correction, specifically on the basis of the previously determined location-dependent correction data.

The imaging mode can also comprise several different visualization modes, each of which is based on different processing of color channel signal values of the at least one image sensor. In this case, it is advantageous if, depending on the selected visualization mode, different color channel signal corrections are applied by the visualization system based on the calibration data in order to calculate suitable modified color channel signal values for the respective visualization mode.

A medical visualization system is also proposed to solve the object mentioned at the outset. This has one or more of the features disclosed herein and is thus characterized in particular in that the visualization system has an image signal processing unit that is designed to perform modified imaging based on calibration data that was previously sensed in a calibration mode using the entire visualization system based on at least one previously performed optical calibration measurement. It is particularly advantageous if the modified imaging is performed on the basis of calibration data that has been determined using a method according as described herein, or has been determined or sensed using the visualization system.

In such a design, it is particularly advantageous for optimum system accuracy if the visualization system is set up to capture the calibration data for (i) different calibration light sub-spectra and/or for (ii) different optical paths and/or for (iii) different color channels of the at least one image sensor and/or for (iv) different color ratios based on different signals of individual color channels of the at least one image sensor (i.e., calculated from these signals by the image signal processing unit). In this way, the system can determine, in particular, spectrally selective and/or path-dependent and/or color channel-dependent and/or color ratio-dependent compensation parameters, which can then be used in imaging mode. With the help of such compensation parameters, the accuracy of imaging and the accuracy in providing enhanced image information can be improved.

Such a visualization system can be implemented, for example, based on a first RGB color image sensor, wherein a beam splitter is used to supply a second, in particular monochrome, image sensor of the system with spectrally selective imaging light.

The at least one image sensor can thus comprise, for example, a color image sensor and/or one or more monochromatic image sensors and/or a region of an active sensor area of an image sensor and/or a line sensor.

For illumination, for example, several LEDs or lasers can be used as respective illumination light sources. Such light sources can be operated together and at a constant rate and/or emit light in a comparatively narrow band around a respective emission wavelength.

The color channels of the image sensor read out for imaging can be formed sensorily by respective pixels of the respective image sensor. For example, three color channels R, G, B could be formed with a conventional RGB image sensor, and at least one additional color channel, for example a second blue color channel or an IR channel, could be created with a second separate image sensor that is supplied with imaging light via a spectrally selective beam splitter.

The system can then determine respective color ratios from the different image signals of the individual color channels of the visualization system, for example for each individual pixel or for pixel areas. Such color ratios can be used, for example, with known algorithms to calculate data that allow conclusions to be drawn about local oxygen saturation or other physiological variables. Such algorithms can be based on non-linear equations, wherein certain (e.g., logarithmic) variables must be determined with high accuracy. The invention is particularly advantageous in such applications precisely because it enables highly accurate determination of color ratios even in the case of manufacturing-related fluctuations in the spectral properties of the visualization system. The invention makes it possible to provide the input data processed by the algorithm with greater accuracy, even if the visualization system used for imaging exhibits typical manufacturing-related fluctuations in its spectral transmission properties or spectral sensitivity. Optical crosstalk between individual color channels of the system can also be compensated for using the approach according to the invention.

However, the approach according to the invention can also be implemented, for example, if the illumination used for imaging (in particular, multiple LEDs) is modulated in time. This latter approach then enables temporal separation of the different spectral components that make up the total spectrum used for imaging or for obtaining additional information. This approach also enables, for example, white light imaging and sub-spectral imaging (e.g., fluorescence imaging) using only a single image sensor.

According to a preferred design, the visualization system has at least five different color channels, each of which can be detected separately by the at least one image sensor; these can preferably be two blue, one green, one red, and one violet color channel or, according to a further design, a combination of two blue, two red, and one NIR color channel. For this purpose, for example, five narrow-band light sources (preferably in the form of LEDs) can be used as illumination light sources in imaging, either modulated in a constant/non-modulated manner (i.e., in particular, all light sources simultaneously) or modulated in time, for example, by alternating illumination with at least two different respective illumination light spectra.

The system may, for example, have a first color image sensor, preferably in the form of an RGB sensor, which provides at least three color channel signals (R/G/B color channel). Furthermore, a second image sensor may be provided with which a sub-spectrum can be detected, which is spatially separated from a first optical path ending at the color image sensor by means of a wavelength-selective optical element (e.g., a wavelength-selective beam splitter).

With such a system design, it is possible to implement a spatially resolved determination (and visualization) of a spatial distribution of a physiological variable such as oxygen saturation SO2(x,y) or a spatial distribution of a specific tissue type (e.g., nerve tissue) based on color channel signal values modified using the method according to the invention. The modified color channel signal values can be sensed by the visualization system with the aid of at least one image sensor, preferably with the aid of two separate image sensors. The determination of such physiological variables can be carried out in particular on the basis of color ratios calculated from such modified color channel signal values by the visualization system, in particular in real time during imaging. The term “color ratios” can be understood here in general as referring to calculated ratios of two color channel signal values, but also to ratios of color channel signal values that have already been calculated with each other. For example, values normalized to a specific color can also be used in the calculation. The term “colors” can also be understood here to mean spectral ranges that lie in the non-visible range, such as in the UV or NIR wavelength range.

A further design may include the use of regression algorithms and/or classification algorithms in the calculation of the color ratios from the respective modified color channel signal values. In such approaches, the respective values may first be normalized to one or more spectral ranges (colors), wherein the corrections described here are applied on the basis of the calibration data.

### BRIEF DESCRIPTION OF THE DRAWINGS

The invention is now described in more detail with reference to exemplary embodiments, but is not limited to these exemplary embodiments. Further embodiments of the invention can be obtained from the following description of a preferred exemplary embodiment in conjunction with the general description, the claims, and the drawings. In the following description of various preferred embodiments of the invention, elements that are identical in function are given identical reference numbers, even if their design or shape differs, wherein:

FIG. 1 shows the possible components of a visualization system according to the invention,

FIG. 2 shows a schematic view of high-precision “advanced visualization” imaging made possible by a visualization system according to the invention,

FIG. 3 shows signal flows within a visualization system according to the invention, as they arise during imaging,

FIG. 4 shows another possible design of a visualization system according to the invention, wherein the signal flows show that the system enables fluorescence imaging and spatially resolved determination of oxygen saturation.

FIG. 5 shows another possible design of a visualization system according to the invention, which, in addition to the functionality of the system shown in FIG. 4, also enables white light imaging simultaneously with fluorescence imaging and spatially resolved determination of oxygen saturation.

FIGS. 6A-6E show spectral characteristics of two optical paths of a visualization system according to the invention and the effects caused by a spectral shift of an illumination light source.

FIGS. 7A-7C show how fluctuating color channel signal values can be compensated for using the calibration method according to the invention, and finally

FIG. 8 shows another possible design of a visualization system according to the invention, wherein, unlike in the example of FIG. 4, different correction values are applied to the individual color channel signal values in order to obtain different modified color channel signal values from the same color channel.

### DETAILED DESCRIPTION

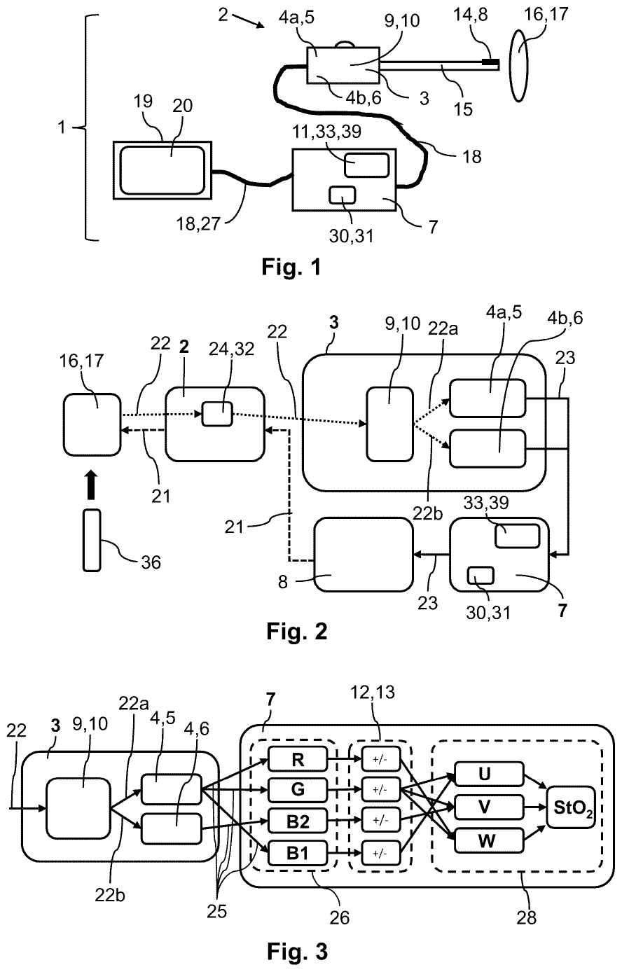

FIG. 1 schematically shows how an object 16, namely human tissue 17, is observed during a medical procedure with a visualization system 1 according to the invention in an imaging mode. The visualization system 1 comprises an endoscope 2 including a camera head 3 with a color image sensor 5 and a monochrome image sensor 6, as well as an illumination unit 14, which preferably has several illumination light sources 8, for example in the form of LEDs, which emit spectrally narrow-band different illumination light spectra, each of which can also be used as calibration light sub-spectra. The system 1 also comprises a camera control unit 7 (CCU), which receives image signals from both image sensors 4a, 4b in the form of raw signals via the illustrated cable 18 and processes these signals via signal processing and then further processes said signals. Specifically, the CCU 7 uses a video chain 11 to generate a video signal 27 from the raw signals and transmits this to the monitor 19 in order to display a live video image 20 of the object 16.

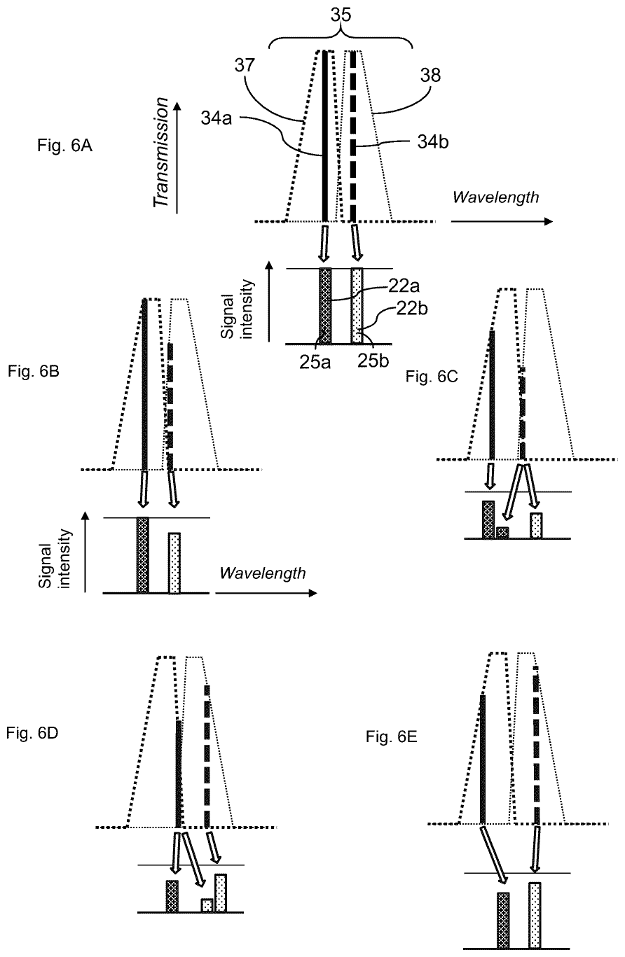

In doing so, the CCU 7, or more precisely its image signal processing unit 33, also calculates extended image information in real time, which is visualized together with the live video image 20. The extended image information is calculated from color channel signal values supplied by the two image sensors 4a, 4b, wherein the color channel signal values are modified on the basis of calibration data 30 which was previously sensed by the system 1 in a calibration mode and stored in a memory 31. For this purpose, several optical calibration measurements are performed with the system 1, each using a specific calibration light sub-spectrum 34, which is detected by the associated color channel 25 to be calibrated (this color channel may, for example, exhibit the highest sensitivity with respect to this calibration light sub-spectrum 34—see, for example, FIGS. 6A-6E).

FIG. 2 shows the system 1 from FIG. 1 in a highly simplified form, focusing on the optical path 21 of the illumination light emitted by the respective light source 8. The optical path 22 relevant for imaging is illustrated with dotted lines and runs from the object 16 observed with the endoscope 2 in imaging mode through an imaging optic 24 of the endoscope 2 and a wavelength-selective optical element 9 (this can be a diffractive element or, for example, a wavelength-selective beam splitter 10 or corresponding filters that are placed in front of the respective image sensor in calibration mode) to the respective image sensor 5/6 in the camera head 3. The optical path 22 is spatially divided into two sub-paths 22a and 22b by the element 9. This also applies when an optical calibration measurement is performed in a calibration mode with the visualization system 1, in which the visualization system 1 detects an optical target 36, as indicated in FIG. 2. In calibration mode, the monochrome image sensor 6 detects a first calibration light sub-spectrum (e.g., invisible wavelengths in the near infrared=NIR) and the color image sensor 5 detects a second calibration light spectrum (e.g., several calibration light sub-spectra in the visible wavelength range), each selectively. In other words, wavelengths detected by the monochrome image sensor 6 do not reach the color image sensor 5 and vice versa. Alternatively, a visualization system 1 according to the invention can also use fiber optics, for example, to guide respective calibration light sub-spectra to the respective image sensors 4a, 4b.

The spatial separation of the individual spectra illustrated in FIG. 2 could also be replaced in a manner known per se by a temporal separation, for example by using temporally alternating spectral illumination of the object 16 or the optical target 36. Alternatively, the spatial separation of the sensor-detected sub-spectra (in the example, VIS and NIR) could also be achieved at the pixel level by using only a single image sensor 4,5, for example on the basis of pixel-based optical filters (as in an R/G/B/IR color image sensor with 4 color channels and R/G/B/IR color filters). This is because the solution concept according to the invention can also be implemented using such approaches.

FIG. 3 shows further details of the exemplary visualization system 1 of FIGS. 1 and 2 (wherein, for reasons of clarity, certain components such as the memory 31 or the computing unit 39 are not illustrated): In this non-limiting example, the abbreviations R/G/B1/B2 stand for a red, a green, a first blue, and a second blue color channel 25 (The visualization system 1 thus offers four different color channels 25), although other color channels can also be used, and the abbreviations U/V/W symbolize respective (exemplary) calculation rules as follows:

$$U = log ( G / B 1 )$$

$$V = log ( B 2 / G )$$

$$W = log ( R / G )$$

During imaging in imaging mode, the CCU 7 therefore calculates logarithmic values of color ratios

$$CR 1 = G ( x , y ) / B 1 ( x , y ) ,$$

$$CR 2 = B 2 ( x , y ) / G ( x , y ) and$$

$$CR 3 = R ( x , y ) / G ( x , y )$$

calculated pixel by pixel, which in turn are calculated as respective ratios of color channel signal values (per pixel) of the respective color channel 25, wherein the color channel signal values are first modified on the basis of calibration data 30 recorded in calibration mode. Here, for example, R(x,y) stands for the color channel signal value of the red color channel 25 at a specific spatial position (x,y) of the active sensor area of the image sensor 4,5 (which corresponds to a specific pixel/spatial position on the observed tissue 17).

The monochrome image sensor 6 provides only one of the four color channels, namely the second blue color channel 25 B2. From these spatially resolved logarithmic color ratios, the CCU 7 (more precisely, its “advanced imaging module” 28) ultimately calculates a spatially resolved spatial distribution StO₂(x,y) of the oxygen saturation StO₂ in the observed tissue 17. The user can display this distribution as a false color overlay on the video image 20, which enables additional diagnostics. This system 1 thus allows oxygen saturation imaging as a possible form of “advanced visualization” or “advanced imaging.”

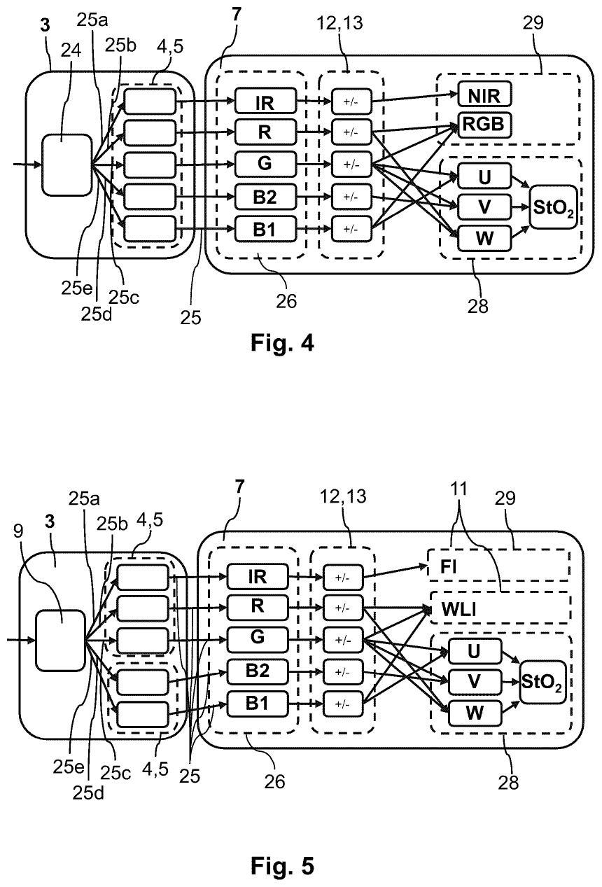

In the example shown in FIG. 4, the visualization system 1 has five color channels IR/R/G/B1/B2, all of which are provided by a single hyperspectral color image sensor 4,5, so that the spatial separation of the respective sub-spectra only takes place at the pixel level and no beam splitter needs to be used. The CCU 7 can thus calculate and output RGB images as well as NIR images based on the color channel signal values provided by the single image sensor 4 in the form of raw signals 26.

In the example shown in FIG. 5, the visualization system 1 also has five color channels 25 IR/R/G/B1/B2, but also two color image sensors 5, wherein the upper first color image sensor 4,5 provides three color channels 25 IR/R/G and the lower second color image sensor 4,5 provides two color channels 25 B1 and B2. Like the systems shown in FIGS. 3 and 4, this system 1 also enables oxygen saturation imaging and can also generate white light images and fluorescence images. To this end, the CCU 7 uses an advanced visualization module (AVM) 29 to perform white light imaging (WLI) and fluorescence imaging (FI) by calculating color channel signal values from individual color channels 25 corresponding to image data.

In a calibration measurement according to the invention, all color channels of the respective image sensor 4 can be read out individually/separately from the other color channels 25, and thus, for each color channel 25, respective spectral compensation data can be sensed with the image sensor 4,5 as calibration data 30. This can be performed for each of the calibration light sub-spectra used.

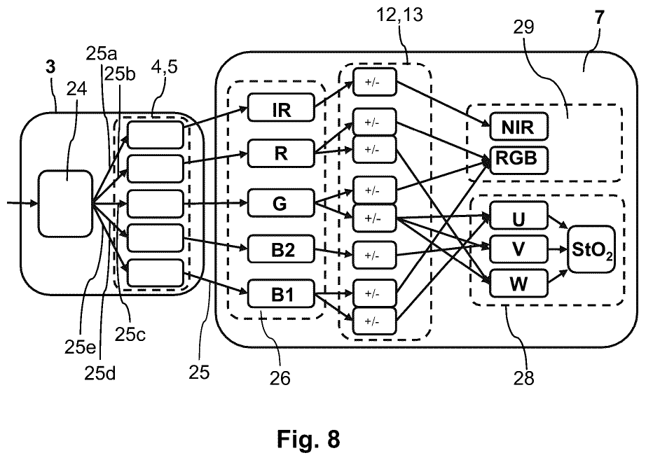

As FIG. 8 shows, based on the calibration data 30, different color channel signal corrections can also be applied to the signal values of the individual color channels IR/R/G/B1/B2, depending on whether a calculated variable U/V/W is determined from the values or a color value for a pixel (e.g., of an NIR or VIS image).

The examples given so far have presented specific processing rules. However, the invention, i.e., the use of color channel signal values modified on the basis of the calibration measurements according to the invention, can be used quite generally in calculations in which color channel signal values obtained from different color channels (in particular from different image sensors of the system) are offset against each other, wherein such an offset may also involve normalization. Furthermore, as mentioned above, modified color ratios can also be obtained from the modified color channel signal values obtained according to the invention if these ratios are formed from color channel signals that are combined and/or already offset against each other. The modified color ratios obtained in this way can then be further processed by another processing unit, wherein, for example, a regression algorithm or a classification algorithm can be used.

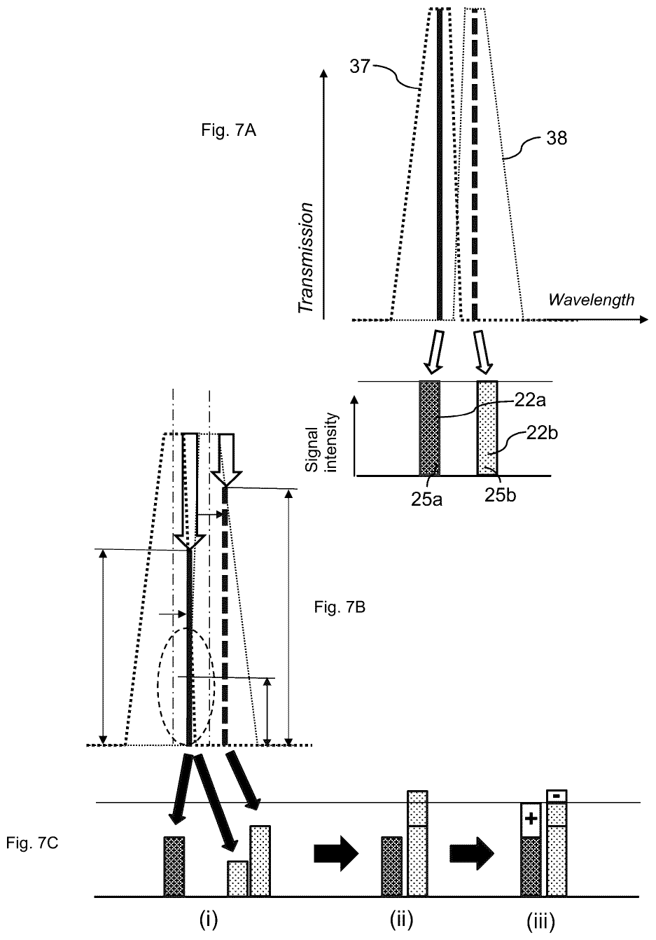

As FIGS. 6A-6E and 7A-7C illustrate, even small manufacturing-related variations (especially when very steep bandpass filters are used in spectrally selective beam splitters) can lead to significant fluctuations in transmission in the optical path used for imaging and thus to errors in the color ratios calculated from the color channel signal values of the image sensor, which are captured by the visualization system 1. This can ultimately result in corresponding measurement errors, i.e., for example, blood oxygen saturation can no longer be determined optically with the visualization system with the desired accuracy of 5% in clinical applications, which of course must be avoided.

FIGS. 6A-6E illustrate the effect when, for example, the illumination light source used exhibits a spectral shift in its emission characteristics: The respective curves represent the wavelength-dependent transmission of the beam splitter in the respective optical path as dotted lines. FIG. 6A shows the case where the emission spectrum of the illumination light source corresponds to the specification and the spectrally selective beam splitter has also been manufactured as specified. In this case, high transmission can be achieved for both optical paths.

FIG. 6B, on the other hand, shows the case where the emission characteristics of the illumination light source shift toward shorter wavelengths (to the left in the figure), and in FIG. 6C, this shift is even greater. FIG. 6D, on the other hand, shows the opposite case, where the emission characteristics shift toward longer wavelengths, and finally, FIG. 6E shows the case where part of the wavelengths emitted by the light source shift toward shorter wavelengths and another part shift toward longer wavelengths, so that the spectrum increases in width. In all these cases, the optical transmission in the respective optical path changes compared to the actual desired situation shown in FIG. 6A. However, these effects can be effectively compensated for using the method according to the invention, e.g., on the basis of modified color ratios, which in turn are based on modified color channel signal values calculated using the calibration data 30 from the corresponding optical calibration measurements.

FIG. 7A shows once again the ideal case in which the spectrally selective beam splitter and the emission characteristics of the illumination light source correspond to the specifications. If the emission spectrum of the light source shifts toward longer wavelengths, as illustrated in FIG. 7B, two effects come into play: First, this results in a loss of signal intensity in both optical paths, as illustrated by the white block arrows in FIG. 7B. However, as can be seen from the dashed ellipse in FIG. 7B, a second effect is the coupling of light into the adjacent optical path (=optical crosstalk), which is actually not intended/undesirable.

As the bar charts (plotting signal intensity vs. wavelength) in FIG. 7C show, this results (compared to the situation in FIG. 7A) in the effect that the signal intensity in the left (dark) optical path is attenuated, while crosstalk into the adjacent optical path causes the signal intensity in this second (light) optical path to increase (compare the diagram in (i)). Without suitable compensation, this would result in signal intensities as illustrated in diagram (ii). However, using the optical calibration method according to the invention, calibration data 30 is collected in calibration mode, which can be used to determine color channel compensation values with which, as shown in diagram (iii), the individual color channel values generated by the respective optical path can be readjusted so that modified color channel signal values or modified image signals can be obtained that correspond to the actual ideal situation in FIG. 7A, i.e., in particular, color ratios can be determined that are less error-prone than before the calibration was performed.

In summary, in order to provide a visualization system 1 that is tolerant of manufacturing-related fluctuations in sensor-detected signal intensities, a method for setting up this visualization system 1 is proposed, which provides for an optical in-situ calibration measurement in which calibration data 30 is sensed using at least one image sensor 4 of the visualization system 1 as part of an optical calibration measurement. Based on this calibration data 30, which is then stored, the visualization system 1 can generate modified color channel signal values that are compensated or modified in such a way that the manufacturing-related fluctuations no longer lead to a distortion of color ratios calculated from the modified color channel signal values. This approach according to the invention thus enables a high degree of accuracy of the signal values and the color ratios calculated from them, in particular in multispectral imaging (FIGS. 7A-7C), where additional calculation steps can also be performed subsequently.

### LIST OF REFERENCE SIGNS

1 Visualization system
2 Endoscope
3 Camera head
4 Image sensor
5 Color image sensor
6 Monochrome image sensor
7 Camera control unit (CCU)
8 Illumination light source
9 Wavelength-selective optical element
10 Wavelength-selective beam splitter
11 Video imaging chain (video chain)
12 Compensation module
13 Calibration block
14 Illumination unit (may comprise one or more 8)
15 Endoscope shaft
16 Object
17 Tissue
18 Cable
19 Monitor
20 Live video image
21 Path of the illumination light (from 8)
22 Optical path used for imaging and/or capturing calibration data (=imaging path)
23 Signal path
24 Imaging optics (comprises several optical components)
25 Color channel (of 4)
26 Raw signals (of 4)
27 Video signal (generated by 1)
28 Advanced imaging module (AIM)
29 Advanced visualization module (AVM)
30 Calibration data
31 Memory (internal or external memory from 1; for storing 30)
32 Optical component (lens, beam splitter, wavelength-selective element, mirror, grating, etc.)
33 Image signal processing unit (may be part of 7)
34 Calibration light sub-spectrum
35 Total spectrum
36 Optical target
37 Transmission characteristic path 1
38 Transmission characteristic path 2
39 Calculation unit (calculates 30; can be part of 33)

### Abbreviations Used

FI=Fluorescence imaging
WLI=White light imaging
AVM=Advanced visualization module
AIM=Advanced imaging module

1. A method for setting up a visualization system (1) for advanced imaging, wherein the visualization system (1) comprises:
at least one image sensor (4), such that the visualization system (1) provides different color channels (25) for multispectral imaging, and
a plurality of optical components (32) which form at least one optical path (22) used for multispectral imaging, the method comprising:
in a calibration mode, performing with the visualization system (1) a number N of optical in-situ calibration measurements, by which calibration data (30) are sensed in each case with the at least one image sensor (4), respectively,
wherein in each of these N calibration measurements, a respective calibration light sub-spectrum (34) is used, which is designed for a respective spectral range to be corrected,
storing the calibration data (30) determined in the calibration mode in an internal or external memory (31) of the visualization system (1), and,
in a subsequent imaging mode,
i) performing a modified extended imaging with the at least one image sensor (4) in which signals from the at least one image sensor (4) are processed in real time to produce live video image data, and,
ii) simultaneously performing a color channel signal correction,
based on the stored calibration data (30) and based on color channel signal values, which the at least one image sensor (4) outputs, and
wherein the color channel signal correction comprises
calculating modified color channel signal values from the color channel signal values and
an extended image information from these modified color channel signal values.

2. The method according to claim 1, wherein the extended image information is visualized together with the produced live video image data on a monitor (19).

3. The method according to claim 1, wherein the calibration data (30) comprise
color channel correction values that describe which target deviation the respective calibration light sub-spectrum (34) generates in an associated one of the color channels (25), and/or
crosstalk correction values that describe spectral crosstalk of the respective calibration light sub-spectrum (34) to an adjacent one of the color channels (25).

4. The method according to claim 1, wherein the calibration data (30) are recorded separately in each case
for different ones of the optical paths (22a, 22b) to calculate path-dependent compensation parameters and/or
for different ones of the color channels (25a, 25b, 25c, 25d, 25e) to calculate color channel-dependent compensation parameters.

5. The method according to claim 1, wherein the imaging mode comprises
performing a sub-spectral signal processing of the modified color channel signal values, by which sub-spectral image information is obtained, which represents only a part of a spectrum that is used for imaging.

6. The method according to claim 5, wherein, as part of said sub-spectral signal processing, an additional information and/or a spatial distribution of a physiological variable is calculated from local color conditions based on the modified color channel signal values, in addition to a first image, which is directly captured by the sensor.

7. The method according to claim 6,
wherein, as part of said sub-spectral signal processing, a sub-spectrum (34), which forms part of a total spectrum (35) sensed by the visualization system (1), is sensed separately in a form of sub-spectral image signals by the at least one image sensor (4), and
wherein, based on the calibration data (30), the sub-spectrum (34) is processed by an image signal processing unit (33) of the visualization system (1) to produce modified sub-spectral image signals.

8. The method according to claim 1, wherein the calibration data (30) describe
wavelength-dependent transmission characteristics of at least one optical path (22) used for imaging within the visualization system (1) and/or
a spectral emission characteristic of an illumination light source (8) used in the subsequent imaging mode.

9. The method according to claim 1, further comprising
in the calibration mode, using at least one calibration light arrangement, which provides a calibration light sub-spectrum (34) whose half-width is limited or actively limited to less than 50 nm, to capture respective spectral calibration data, which are spectrally selectively for several different non-overlapping spectral ranges.

10. The method according to claim 9, wherein the at least one calibration light arrangement is realized as a passive optical target (36), which, when irradiated with suitable light, re-emits a spectrum with a half-width of less than 50 nm.

11. The method according to claim 1, the method further comprising, in the imaging mode, determining numerical color channel compensation values from the calibration data (30), with which different color channel signal values output by the at least one image sensor (4) are each converted into modified color channel signal values, and determining at least one modified color ratio.

12. The method according to claim 1, further comprising, in the calibration mode, recording spectral transmission data as part of said calibration data (30) from at least one optical path (22), which is used for imaging in the imaging mode.

13. The method according to claim 1, further comprising, in the calibration mode, detecting, as part of said calibration data (30), a spectral emission characteristic of an illumination light source (8) used in the subsequent imaging mode,
wherein said spectral emission characteristic is detected by using a calibration light sub-spectrum (34), which is emitted by at least one wavelength-selective optical element.

14. The method according to claim 1, wherein the visualization system (1) comprises an endoscope (2) or an exoscope and
wherein, in the calibration mode,
an optical calibration device specially designed for the endoscope (2) or the exoscope is used, and
wherein the optical calibration device remains arranged on a shaft of the endoscope (2), while the optical calibration measurement is performed with the visualization system (1).

15. The method according to claim 1, further comprising, when determining the modified image signals in the imaging mode, taking into account
a) at least one color channel-specific color compensation value, which is based on the calibration data (30), and/or
b) at least one color channel-specific crosstalk correction value, which is based on the calibration data (30).

16. The method according to claim 1, further comprising,
in the calibration mode, recording the calibration data (30) in a spatially resolved manner for different image areas and/or for different pixels and storing the calibration data (30) as location-dependent correction data, and,
in the imaging mode and as part of said color channel signal correction, modifying the color channel signal values for different pixels differently.

17. The method according to claim 1,
wherein the imaging mode comprises several different visualization modes, wherein each visualization mode is based on a different processing of color channel signal values of the at least one image sensor (4), and
the method further comprising, depending on a selected visualization mode, applying different color channel signal corrections by the visualization system (1) based on the calibration data (30) in order to calculate suitable modified color channel signal values for the respective visualization mode.

18. A medical visualization system (1), comprising:
at least one image sensor (4),
a plurality of optical components (32) that define at least one optical path (22) used for imaging,
an image signal processing unit (33) configured to perform a modified extended imaging,
based on calibration data (30), which were previously sensed in a calibration mode with the visualization system (1), using the method according to claim 1.

19. The medical visualization system (1) according to claim 18, wherein the image signal processing unit (33) is further configured to separately record the calibration data (30) in each case for at least one of:
a) different calibration light sub-spectra,
b) different optical paths (22a, 22b),
c) different color channels (25a, 25b, 25c, 25d, 25e) of the at least one image sensor (4), or
d) different color ratios, which result from different signals of individual color channels (25a, 25b, 25c, 25d, 25e) of the at least one image sensor (4).

20. The medical visualization system (1) of claim 18, wherein the medical visualization system (1) is implemented in the form of an endoscope, a microscope, or an exoscope.
